(19) United States
(12) Patent Application Publication
(10) Pub. No.: US 2012/0308669 A1
(43) Pub. Date: Dec. 6, 2012

(54) CHOCOLATE CANDIES FORTIFIED WITH NATURAL AMINO ACIDS AND/OR HERBAL NUTRIENTS FOR RELIEF OF INSOMNIA, PMS, AND DIFFICULTY CONCENTRATING

(76) Inventors: Gerald Zachary Smith, JR., Saint Francisville, LA (US); Catherine Corbett Smith, Saint Francisville, LA (US)

(21) Appl. No.: 13/485,864

(22) Filed: May 31, 2012

Related U.S. Application Data
(60) Provisional application No. 61/491,718, filed on May 31, 2011.

(51) Int. Cl. A61K 36/00; A61P 15/00; A61P 43/00; A61K 33/18

(52) U.S. Cl. 424/667; 424/725

(57) ABSTRACT

A confection, such as chocolate, can be formulated to include naturally occurring amino acids and/or herbal extracts and/or plant extracts incorporated therein. The formulation allows a consumer to relieve health issues in a convenient, enjoyable manner. By incorporating the appropriate amino acids/herbal and plant extracts into a chocolate candy form, the confections can be formulated to provide relief of various ailments, such as insomnia, fatigue, PMS, difficulty concentrating and the like.

### CROSS-REFERENCE TO RELATED APPLICATIONS

This application claims the benefit of provisional patent application No. 61/491,718, filed May 31, 2011, the contents of which are herein incorporated by reference.

### BACKGROUND OF THE INVENTION

The present invention relates to nutritional supplements and, more particularly, to chocolate candies fortified with natural amino acids and/or herbal nutrients for relief of insomnia, premenstrual syndrome (PMS), and difficulty concentrating.

Many individuals suffer from insomnia, fatigue and other issues such as PMS and the lack of mental focus. Many individuals do not like taking pills or capsules, and others are not able to consume grain-based products due to digestive issues. Moreover, it is not always easy to remember to take supplemental nutrients, nor is it always convenient to take twenty or more supplement pills or capsules in the course of a day.

As can be seen, there is a need for an improved formulation and method for providing users with supplemental nutrients that does not involve taking pills or capsules and can be easy to remember, easy to consume, and reduces or eliminates the need to take pill or capsule-based supplements.

### SUMMARY OF THE INVENTION

In one aspect of the present invention, a therapeutic chocolate confection comprises at least one of one or more natural amino acids and one or more herbal nutrients adapted for the relief of at least one of insomnia, fatigue, premenstrual syndrome, stress and difficulty concentrating.

In another aspect of the present invention, a method for the treatment of at least one of insomnia, fatigue, premenstrual syndrome, stress and difficulty concentrating comprises administering a therapeutically effective amount of a chocolate confection comprising at least one of one or more natural amino acids and one or more herbal nutrients.

These and other features, aspects and advantages of the present invention will become better understood with reference to the following description and claims.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a confection, such as chocolate, having naturally occurring amino acids and/or herbal extracts and/or plant extracts incorporated therein. The formulation of the present invention allows a consumer to relieve health issues in a convenient, enjoyable manner. By incorporating the appropriate amino acids/herbal and plant extracts into a chocolate candy form, the confections of the present invention can be formulated to provide relief of various ailments, such as insomnia, fatigue, PMS, difficulty concentrating and the like.

Embodiments of the present invention include (1) chocolate in confection form used as a delivery system for combinations of gamma-aminobutyric acid, 5-hydroxytryptophan, L-glutamine, melatonin, gelatin, other naturally occurring amino acids, and herbal extracts in order to promote sleep; (2) chocolate in confection form used as a delivery system for combinations of L-tyrosine, L-glutamine, iodine in mineral form, and herbal extracts in order to promote higher energy levels and combat fatigue; and (3) chocolate in confection form is used as a delivery system for combinations of 5-hydroxytryptophan, L-glutamine, gamma-aminobutyric acid, and herbal extracts in order to combat premenstrual syndrome and stress.

For the promotion and enhancement of sleep: gamma-amino butyric acid is the chief inhibitory neurotransmitter in mammals, including and especially man. The use of this naturally occurring amino acid has a well-established relaxing effect, which aids in calming at night when sleep is desired. 5-Hydroxytryptophan (5-HTP) is produced in the body from L-tryptophan, an essential amino acid derived from animal and plant proteins. 5-HTP is converted by the body to 5-HT, 5-hydroxytryptamine, commonly known as serotonin. Serotonin is preferentially converted by the body to melatonin when it is time for sleep. Gelatin primarily consists of glycine, which has calming properties similar to GABA. It can help to induce relaxation in the neurological system. L-taurine has inhibitory properties similar to GABA, and can help to induce relaxation in the neurological system.

For relief of fatigue: L-tyrosine is a non-essential amino acid produced by the body from L-phenylalanine. L-tyrosine is further converted, along two separate metabolic pathways, to dopamine or thyroxin, or T-4, one of the thyroid hormones. Dopamine is essential for regulation of cognition and motor activities, and produces a state of enhanced motivation for performance. T-4 is converted to T-3, which is the chief regulator of metabolism and enhances energy production. Iodine, in mineral form, is required to produce T-4 from L-tyrosine.

For relief of symptoms of premenstrual syndrome: GABA produces relaxation throughout the body. 5-HTP produces a sense of wellbeing, enhancing serotonin levels. L-taurine produces relaxation throughout the body. L-glutamine may be utilized directly by the brain for production of energy in the brain cells. It can thus enhance brain function and facilitate utilization of GABA and 5-HTP.

For enhanced concentration: L-phenylalanine and L-tyrosine enhance mental focus and hence concentration. D,L-phenylalanine, as a naturally occurring mixture, provides L-phenylalanine which enhances mental focus and hence concentration. L-glutamine may be utilized directly by the brain for production of energy in the brain cells. It thus enhances mental focus and the ability to concentrate.

For sleep, a person would consume a therapeutically effective amount of the product to enhance and promote sleep. A recommended serving will be suggested, but the consumer is free to adjust this as necessary.

For relief of fatigue, a person would consume a therapeutically effective amount of the product to enhance and promote higher energy levels. A recommended serving will be suggested, but the consumer is free to adjust this as necessary.

For relief of PMS, a woman would consume a therapeutically effective amount of the product to produce a calming effect. A recommended serving will be suggested, but the consumer is free to adjust this as necessary.

For improved concentration, a person would consume a therapeutically effective amount of the product to enhance and improve focus and mental clarity. A recommended serving will be suggested, but the consumer is free to adjust this as necessary.

To make the chocolate confections of the present invention, chocolate of any type, including but not limited to dark, milk and white, may be melted using commonly known techniques. Amino acids, herbal nutrients and appropriate mineral supplements, typically in powder form, are then added to the chocolate while it is in the molten state. This mixture is stirred or otherwise mixed. The chocolate is then placed into molds and allowed to cool, producing the chocolate candy containing the desired supplements. The chocolate candies of the present invention may be bite-sized snacks that provide snacking enjoyment while also providing a health benefit.

While the above describes confections containing supplements, the present invention may also be provided in a liquid form—either as a ready to drink supplement, or as an additive to be added to a base material, such as water or milk, for example.

It should be understood, of course, that the foregoing relates to exemplary embodiments of the invention and that modifications may be made without departing from the spirit and scope of the invention as set forth in the following claims.

What is claimed is:

1. A therapeutic chocolate confection comprising:
at least one of one or more natural amino acids and one or more herbal nutrients adapted for the relief of at least one of insomnia, fatigue, premenstrual syndrome, stress and difficulty concentrating.

2. The therapeutic chocolate confection of claim 1, wherein the natural amino acid includes L-tyrosine and L-glutamine and the therapeutic chocolate confection includes iodine in mineral form and the therapeutic chocolate confection is adapted for the relief of fatigue.

3. The therapeutic chocolate confection of claim 1, wherein the natural amino acid includes 5-hydroxytryptophan, L-tryptophan, L-taurine, L-glutamine, and gamma-amino butyric acid and the therapeutic chocolate confection is adapted for combating premenstrual syndrome and stress.

4. The therapeutic chocolate confection of claim 1, wherein the natural amino acid includes L-tyrosine, L-glutamine, and at least one of L-phenylalanine and D,L-phenylalanine and the therapeutic chocolate confection is adapted for the relief of difficulty concentrating.

5. The therapeutic chocolate confection of claim 1, wherein the confection is formulated as a bite-sized solid chocolate confection snack.

6. A method for the treatment of at least one of insomnia, fatigue, premenstrual syndrome, stress and difficulty concentrating comprising administering a therapeutically effective amount of a chocolate confection comprising at least one of one or more natural amino acids and one or more herbal nutrients.

7. The method of claim 6, wherein:
the natural amino acid includes L-tyrosine and L-glutamine;
the chocolate confection includes iodine in mineral form; and
the therapeutic chocolate confection is adapted for the relief of fatigue.

8. The method of claim 6, wherein:
the natural amino acid includes 5-hydroxytryptophan, L-tryptophan, L-taurine, L-glutamine, and gamma-amino butyric acid; and
the therapeutic chocolate confection is adapted for combating premenstrual syndrome and stress.

9. The method of claim 6, wherein:
the natural amino acid includes L-tyrosine, L-glutamine, and at least one of L-phenylalanine and D,L-phenylalanine; and
the therapeutic chocolate confection is adapted for the relief of difficulty concentrating.

10. The method of claim 6, wherein the confection is formulated as a bite-sized solid chocolate confection snack.
